(19) United States
(12) Patent Application Publication
(10) Pub. No.: US 2026/0248992 A1
(43) Pub. Date: Aug. 27, 2026

(54) METHODS AND DEVICE RELATED TO REMOVAL OF CELL-FREE PLASMA HEMOGLOBIN

(71) Applicant: UNIVERSITY OF PITTSBURGH-OF THE COMMONWEALTH SYSTEM OF HIGHER EDUCATION, Pittsburgh, PA (US)

(72) Inventors: Hulya BAYIR, Pittsburgh, PA (US); Nahmah KIM-CAMPBELL, Pittsburgh, PA (US); Ryan ORIZONDO, Pittsburgh, PA (US); William R. WAGNER, Gibsonia, PA (US); Madison Lee BUTLER, Pittsburgh, PA (US); William J. FEDERSPIEL, Tampa, FL (US)

(21) Appl. No.: 18/993,816

(22) PCT Filed: Jul. 13, 2023
(86) PCT No.: PCT/US2023/027627

§ 371 Date: Jan. 13, 2025

Related U.S. Application Data
(60) Provisional application No. 63/388,805, filed on Jul. 13, 2022.

(51) Int. Cl. A61M 1/36; A61M 1/16; A61M 1/34; A61M 60/113; A61M 60/117; A61M 60/279; A61M 60/37; A61M 60/38

(52) U.S. Cl. CPC A61M 1/3679; A61M 1/1698; A61M 1/3496; A61M 1/362264; A61M 1/3687; A61M 60/113; A61M 60/117; A61M 60/279; A61M 60/37; A61M 60/38; A61M 2202/0415

(57) ABSTRACT

An example system includes a purification chamber including a purification chamber including a purification column, where the purification column includes a capture structure, where a ligand is conjugated on a surface of the capture structure, where the In ligand is specific for a component to be removed from a fluid of a subject; and a pressure source configured to circulate the fluid from a subject through the purification chamber. An example method includes removing blood from a subject; and exposing the blood to a purification column, where the purification column includes at least one capture structure, where a ligand is conjugated on a surface of said capture structure, where the ligand is specific for a component to be removed from the blood of the subject, thereby removing said component from the subject's blood; and returning the blood to the subject.

### BACKGROUND

Hemolysis and increased levels of cell-free plasma hemoglobin (PHb) are common in extracorporeal therapies (ECT) such as cardiopulmonary bypass (CPB) and extracorporeal membrane oxygenation (ECMO)³³⁻³⁵. PHb toxicity can have detrimental effects on the vasculature as well as several organ systems³⁶ with levels greater than ~50 mg/dL during ECMO associated with >4-fold increased odds of mortality^{4,37}. The international consortium, the Extracorporeal Life Support Organization, has guidelines that define a PHb level of 50 mg/dL during ECMO as actionable. Even in relatively low-risk patients, 55% have a PHb level>50 mg/dL at the end of CPB. Although PHb levels decrease over 24 hours after CPB, even this short-lived exposure is clinically relevant with higher levels being a risk factor for acute kidney injury (AKI)²⁰.

PHb has been identified as a risk factor for the AKI that occurs after ECT^{19,28}. PHb can filter through the glomerulus and be actively endocytosed by and have toxic effects on renal proximal tubular epithelial cells²⁹⁻³². AKI is a common complication of CPB and affects up to 52% of post-operative patients¹⁻³. CPB-associated AKI is associated with >2-fold increase in early mortality in adults⁷ while critically ill children with AKI have>3-fold increased odds of mortality and up to 4-fold increase in ICU length of stay (LOS)⁸⁻¹³. Even in the subpopulation of pediatric patients undergoing CPB that had no mortality, AKI had an impact on healthcare resources and cost as these patients had longer ICU and hospital LOS²⁰. Perioperative strategies to prevent AKI reduce mortality and LOS¹⁴⁻¹⁶ making AKI prevention an obvious clinical priority. Yet, although PHb's role in AKI after ECT is widely accepted, there are no known interventions in clinical practice in this country that target PHb production.

PHb exerts its effects through nitric oxide (NO) bioavailability and oxidative stress. NO is responsible for the maintenance of vascular tone³⁸ and its bioavailability results, in part, from a balance between endothelial generation and scavenging by Hb, which occurs 1000 times faster with extracellular PHb^{39,40}. PHb-related decreases in NO bioavailability result in vasoconstriction^{21,22} in sickle cell disease and are associated with endothelial activation, brain injury, and death in patients with malaria²⁴. ECT can lead to PHb levels higher than the upper limit measured during vaso-occlusive crisis in sickle cell patients (32 mg/dL) and the level associated with an 80% reduction of forearm blood flow in response to nitroprusside (9.7 mg/dL)^{20,22,41}. In hemodialysis, another form of ECT, increased PHb is associated with a>70% decrease in NO bioavailability and is inversely correlated with arterial flow-mediated dilation⁴².

PHb also plays a role in systemic oxidative stress. In the presence of H₂O₂, circulating PHb functions as a peroxidase leading to intra- and extra-cellular oxidative stress^{23,43,44}. This enzymatic activity can lead to the ongoing consumption of NO, which also acts as a reductant that reduces the ferryl Fe⁴⁺ heme⁴⁵. Binding of PHb with haptoglobin (Hp) decreases its peroxidase activity, however, circulating PHb-Hp complexes can also contribute to oxidative injury²³ and rely on the reticuloendothelial system for clearance from the circulation. The oxidative potential of PHb and PHb-Hp complexes and depletion of antioxidants/reducing agents can then lead to increased levels of protein, lipid, and DNA oxidation products. Levels of PHb have been correlated with measures of increased lipid oxidation in adults with AKI after CPB¹⁸. Pediatric CPB data showed increased lipid oxidation products (9- and 13-hydroxyoctadecadienoic acid [HODEs]) that are associated with the production of PHb and have vasoactive effects²⁷.

The toxic effects of PHb are unfortunately not limited to its tetrameric or heterodimeric forms, however, and extend to its degradation products, including hemin³⁶. The multifactorial aspect of the toxicities of PHb has hindered the development of a single definitive therapy that can eradicate its harmful effects. Thus, PHb itself rather than its degradation products or effects is the most meaningful therapeutic focus in this pathologic continuum given its position as the most upstream target.

There have been a few developments in the field of blood purification. In general, the therapies are nonspecific and have only shown efficacy in vitro or had varying success in small clinical trials. Dialysis and hemofiltration ubiquitously remove solutes under a certain molecular weight (MW) and those used most commonly do not remove PHb. High-flux membranes of extended permeability or high-cutoff membranes have shown some ability in vitro to remove PHb⁴⁶. However, PHb has a relatively large MW (~64kD) which would suggest that a membrane permeable to PHb is also permeable to other potentially beneficial or essential plasma proteins. The Cytosorb® extracorporeal cytokine adsorber is composed of a porous polymer bead platform that binds a range of substances from whole blood and plasma via pore capture and surface adsorption. This device has undergone pilot trials with varying efficacy^{47,48} and is, again, nonspecific in its removal. Its MW-based filtration approach results in relatively minor and variable PHb removal as well as the likely removal of other unknown, potentially beneficial, blood components. In clinical practice, strategies to reduce PHb levels are limited and include mechanical adjustments to circuit components or cannulas. When this fails, plasma exchange is sometimes used and involves plasma replacement. This therapy has been described in case reports during therapy with ventricular assist devices^{49,50} and can be used for severe hemolysis during ECMO. Plasma exchange requires close monitoring, requires continuous calcium replacement, and alters the coagulation profile. Both plasma exchange and dialysis require the use of additional, often limited, resources (staffing to run pumps, circuits and circuit components, donor plasma, calcium infusions) and is not readily available in all centers. These methods are either resource heavy and/or inescapably nonspecific which is undesirable, especially in the care of the unstable and fragile critically ill patient needing ECT. Thus, an easily implementable and highly specific means to target PHb removal will not only be more accessible but will also have a better safety profile.

There has been some development in the use of Hp therapeutics. Hp therapy attenuated the renal dysfunction associated with transfusion-related hemolysis in guinea pigs⁵¹. In a few small clinical studies, the use of intravenous Hp therapy prevented renal injury after CPB^{52,53}, supporting the rationale of this particular strategy of targeting PHb. Circulating Hb-Hp complexes, however, can form aggregates that retain peroxidase activity, remain as a source of oxidative stress, and cause macrophage cytotoxicity²³, especially in the inflammatory milieu common during ECT. Thus, this can potentially have troublesome downstream effects on immune function and the exposed endothelium.

What is needed in the art is a device with an immobilized ligand specific for a target such as hemoglobin. For example, in the case of hemoglobin, the ligand can be haptoglobin. This needed device harnesses the benefits of haptoglobin therapy while preventing the unwanted exposure to hemoglobin-haptoglobin complexes and aggregates.

### SUMMARY

In some aspects, the techniques described herein relate to an extracorporeal hemoadsorption device including: a purification column, wherein said purification column includes a capture structure, wherein a ligand is conjugated on a surface of said capture structure, wherein said ligand is specific for a component to be removed from blood of the subject.

In some aspects, the techniques described herein relate to a device, wherein said device is connected to a device for extracorporeal therapy, wherein said extracorporeal therapy removes fluid from a subject and returns fluid to the same subject,

In some aspects, the techniques described herein relate to a device, wherein the extracorporeal therapy is an extracorporeal life support (ECLS) system.

In some aspects, the techniques described herein relate to a device, wherein the ECLS system includes a pump.

In some aspects, the techniques described herein relate to a device, wherein the device for extracorporeal therapy is a cardiopulmonary bypass system.

In some aspects, the techniques described herein relate to a device, wherein the device for extracorporeal therapy is an extracorporeal membrane oxygenation (ECMO) system.

In some aspects, the techniques described herein relate to a device, wherein the device for extracorporeal therapy further includes an oxygenator.

In some aspects, the techniques described herein relate to a device, wherein the device for extracorporeal therapy further includes a venous reservoir.

In some aspects, the techniques described herein relate to a device, wherein the device for extracorporeal therapy is a ventricular assist device.

In some aspects, the techniques described herein relate to a device, wherein the device for extracorporeal therapy involves plasma exchange.

In some aspects, the techniques described herein relate to a device, wherein the device for extracorporeal therapy is a renal replacement therapy device.

In some aspects, the techniques described herein relate to a device, wherein the capture structure is a porous bead.

In some aspects, the techniques described herein relate to a device, wherein the porous bead is between 50 and 500 μm.

In some aspects, the techniques described herein relate to a device, wherein the bead further includes a polymer attached to the bead.

In some aspects, the techniques described herein relate to a device, wherein the polymer is polyethylene glycol (PEG).

In some aspects, the techniques described herein relate to a device, wherein the specific component to be removed includes a protein.

In some aspects, the techniques described herein relate to a device, wherein the protein is cell-free plasma hemoglobin.

In some aspects, the techniques described herein relate to a device, wherein the ligand is zinc, haptoglobin, or hemoglobin-binding protein (HBP).

In some aspects, the techniques described herein relate to a device, wherein said protein is an antibody, cytokine, or toxin.

In some aspects, the techniques described herein relate to a device, wherein the component to be removed from the blood is a virus or bacteria.

In some aspects, the techniques described herein relate to a device, wherein the device is configured so that blood enters the hemoadsorption device at a proximal end and blood exits the hemoadsorption device at a distal end.

In some aspects, the techniques described herein relate to a device, wherein said proximal end of the hemoadsorption device is in fluid communication with a pump.

In some aspects, the techniques described herein relate to a device, wherein the porous bead is incorporated in a cartridge.

In some aspects, the techniques described herein relate to a device, wherein said device includes an integrated flow sensor.

In some aspects, the techniques described herein relate to a device, wherein said device includes an integrated pressure differential sensor.

In some aspects, the techniques described herein relate to a device, wherein the device further includes an integrated mechanism to adjust resistance to allow modification of blood flow through the device.

In some aspects, the techniques described herein relate to a device, wherein the device includes one or more sensors used to measure cell-free hemoglobin.

In some aspects, the techniques described herein relate to a device, wherein the device measures overall systemic levels of cell-free plasma hemoglobin.

In some aspects, the techniques described herein relate to a device, wherein the device measures overall saturation of cell-free plasma hemoglobin within the device, thereby requiring a cartridge change.

In some aspects, the techniques described herein relate to a method for removing a target component from a subject, the method including: (a) removing blood from a subject; and (b) exposing said blood from step a) to a purification column, wherein said purification column includes at least one capture structure, wherein a ligand is conjugated on a surface of said capture structure, wherein said ligand is specific for a component to be removed from the blood of the subject, thereby removing said component from the subject's blood; and (c) returning the blood to the subject.

In some aspects, the techniques described herein relate to a method, wherein said blood is removed and returned to the subject via a pump.

In some aspects, the techniques described herein relate to a method, wherein said pump is part of an extracorporeal therapy system.

In some aspects, the techniques described herein relate to a method, further including oxygenating the blood.

In some aspects, the techniques described herein relate to a method, wherein the method further includes passing the blood through a venous reservoir.

In some aspects, the techniques described herein relate to a method, wherein the extracorporeal therapy system is a cardiopulmonary bypass system.

In some aspects, the techniques described herein relate to a method, wherein the extracorporeal therapy system is a ventricular assist device.

In some aspects, the techniques described herein relate to a method, wherein the extracorporeal therapy system is a renal replacement therapy device.

In some aspects, the techniques described herein relate to a method, wherein the capture structure is a porous bead.

In some aspects, the techniques described herein relate to a method, wherein the bead is between 50 and 500 μm.

In some aspects, the techniques described herein relate to a method, wherein the bead further includes a polymer attached to the bead.

In some aspects, the techniques described herein relate to a method, wherein the polymer is polyethylene glycol (PEG). 42.

In some aspects, the techniques described herein relate to a method, wherein the target to be removed is a protein.

In some aspects, the techniques described herein relate to a method, wherein the protein to be removed is cell-free plasma hemoglobin.

In some aspects, the techniques described herein relate to a method, wherein the ligand is zinc, haptoglobin, or hemoglobin-binding protein (HBP).

In some aspects, the techniques described herein relate to a method, wherein the protein is an antibody, cytokine, or toxin.

In some aspects, the techniques described herein relate to a method 30-45, wherein the component to be removed from the blood is a virus or bacteria.

In some aspects, the techniques described herein relate to a system including: a purification chamber including: a purification column, wherein said purification column includes a capture structure, wherein a ligand is conjugated on a surface of said capture structure, wherein said ligand is specific for a component to be removed from a fluid of a subject; and a pressure source configured to circulate the fluid from a subject through the purification chamber.

In some aspects, the techniques described herein relate to a system, wherein the purification chamber further includes an inlet and an outlet, wherein the fluid flows from the outlet to the inlet.

In some aspects, the techniques described herein relate to a system, further including a sensor configured to measure a property of the fluid.

In some aspects, the techniques described herein relate to a system, wherein the property of the fluid is a hemoglobin concentration of the fluid.

In some aspects, the techniques described herein relate to a system or claim 50, wherein the sensor is configured to measure absorbance at approximately 540 nm.

In some aspects, the techniques described herein relate to a system, wherein the sensor is an optical sensor configured to measure a property of the fluid entering the inlet of the purification chamber.

In some aspects, the techniques described herein relate to a system, wherein the sensor is an optical sensor configured to measure a property of the fluid entering the outlet of the purification chamber.

In some aspects, the techniques described herein relate to a system, further including a regeneration chamber in fluid communication with the purification chamber.

In some aspects, the techniques described herein relate to a system, wherein the regeneration chamber includes guanidine.

In some aspects, the techniques described herein relate to a system further wherein the regeneration chamber includes a removable cartridge.

In some aspects, the techniques described herein relate to a system, further including a bypass conduit in fluid communication with the pressure source and the subject. 59.

In some aspects, the techniques described herein relate to a system, further including an oxygenator in fluid communication with the pressure source.

In some aspects, the techniques described herein relate to a system, further including a venous reservoir in fluid communication with the pressure source. Other systems, methods, features and/or advantages will be or may become apparent to one with skill in the art upon examination of the following drawings and detailed description. It is intended that all such additional systems, methods, features and/or advantages be included within this description and be protected by the accompanying claims.

### DESCRIPTION OF DRAWINGS

FIG. 1A-G shows (A) a schematic showing the PHb removal device implemented within a CPB support circuit. (B) shows a schematic PHb removal device as used in a patient. (C) shows a PHb removal device showing the flow of blood and porous bead design. (D) Predicted PHb during pediatric CPB. The dotted line represents the case of no PHb removal therapy whereas solid lines represent predictions for varying device flow rates (Q) and removal efficiencies (E). Patient and circuit blood volumes of 85 mL/kg and 150 mL, respectively, were assumed. (E) shows a schematic of a PHb removal device configured to be recharged by a removable cartridge; (F) shows a schematic of a PHb removal device configured to be recharged by a removable cartridge placed in line with the PHb removal device; (G) shows a schematic of an PHb removal device configured for ECMO and/or ECLS.

FIG. 2A-C shows (A) Bound Hp and PHb binding capacity increased with amount of Hp added during bead modification. Measurements were made in vitro in plasma from hemolyzed rat blood. (B) Hp-modified beads (left) exhibited clear red color change due to PHb binding; (C) shows PHb over time both with and without PHb filtration.

FIG. 3A-B shows (A) Trans-column pressure difference was constant during blood recirculation. (B) Columns during blood filling (left) and after rinsing with saline following two hours of blood recirculation (right).

FIG. 4A-B shows (A) Blood velocity showing uniform blood flow at the column entrance. (B) Experimental setup used during blood studies characterizing PHb binding.

FIG. 5A-C shows (A) PHb increases during rat ECT and returns to baseline by 24 h post reperfusion (REP). (B) Serum creatinine increases and remains elevated during and after ECT and is correlated with a (C) change in PHb, R2=0.86, p<0.01. *p<0.05. n=5 for all plots.

FIG. 6A-G shows Prussian Blue staining (blue) (A) is greater in tubular epithelial cells of rats undergoing ECT (C) vs. naive (B). The number of positive TUNEL stained cells (brown) (D) is higher in rats undergoing ECT (F) vs. naive (E). Caspase activity is higher in rats undergoing ECT vs. naive (G). Median (IQR).

FIG. 7 shows a table of experimental groups used to test an experimental embodiment of the present disclosure.

FIG. 8 shows an example computing device.

### DETAILED DESCRIPTION

### General Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces bacterial growth” means reducing the rate of growth of a bacteria relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, chickens, ducks, geese, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “perfusion”, as used herein, refers to the process of a body delivering blood to a capillary bed in its biological tissue.

The term “perfusion pump”, as used herein, refers to a device for simulating cardiopulmonary function.

The term “perfusion circuit”, as used herein, refers to a perfusion system for the extracorporeal preservation of vitality or regeneration of organs, limbs or other biological tissue.

The term “infusion pump”, as used herein, refers to a medical device that delivers fluids, such as nutrients and medications, into a patient's body in controlled amounts. Infusion pumps are in widespread use in clinical settings such as hospitals, nursing homes, and in the home. An infusion pump infuses fluids, medication or nutrients into a patient's circulatory system. It is generally used intravenously, although subcutaneous, arterial and epidural infusions are occasionally used.

The term “fluid”, as used herein, refers to a substance that continually deforms (flows) under an applied shear stress. In biological terms, a fluid is generally blood, a growth medium, lymph fluid, therapeutic agents in liquid, or other biological fluids. The present invention is not to be limited by the nature of the fluid. In some embodiments, fluid may comprise a plurality of small solid elements which may respond to pumping as a fluid. In some embodiments, a fluid may comprise a mixture of fluid and solid elements. In some embodiments, a fluid comprises viscous solutions, solid laden liquids, slurries or pastes.

The term “cardiovascular system”, as used herein, refers to an organ system that permits blood to circulate and transport nutrients (such as amino acids and electrolytes), oxygen, carbon dioxide, hormones, and blood cells to and from the cells in the body to provide nourishment and help in fighting diseases, stabilize temperature and pH, and maintain homeostasis.

The term “extracorporeal”, as used herein, refers to being situated outside the body, such as the body of a subject or the bounds of a living system.

The term “medical device”, as used herein, refers broadly to any apparatus used in relation to a medical procedure. Specifically, any apparatus that contacts a patient during a medical procedure or therapy is contemplated herein as a medical device. Similarly, any apparatus that administers a compound or drug to a patient during a medical procedure or therapy is contemplated herein as a medical device.

“Direct medical implants” or “surgical devices” include, but are not limited to, ventricular assist devices, infusion pump, feeding pump, extracorporeal pump, perfusion pumps, extracorporeal membrane oxygenators, urinary and intravascular catheters, dialysis shunts, wound drain tubes, skin sutures, vascular grafts and implantable meshes, intraocular devices, implantable drug delivery systems and heart valves, and the like.

### Description of Device and Methods of Use

Disclosed herein is an extracorporeal hemoadsorption device comprising: a purification column, wherein said purification column comprises a capture structure, wherein a ligand is conjugated on a surface of said capture structure, wherein said ligand is specific for a component to be removed from blood of the subject. The extracorporeal hemoadsorption device described herein can be connected to (i.e., integrated with) a device for extracorporeal therapy, wherein said extracorporeal therapy removes fluid from a subject and returns fluid to the same subject. The possibilities of use for clinical purposes of techniques for extracorporeal treatment of blood are extremely varied; they comprise a wide group of therapies which include intermittent or continuous renal support, apheresis and hemoperfusion treatment for removal of cytokines or toxins, the various techniques of extracorporeal support for the vital functions (known also as Extracorporeal Life Support or ECLS) including Extracorporeal Membrane Oxygenation or ECMO, veno-arterial for cardiac support or veno-venous for respiratory support, such as a cardiopulmonary bypass system, and the techniques for extracorporeal removal of carbon dioxide (ECCO₂R). The extracorporeal therapy system can optionally comprise elements such as an oxygenator, a pump, or a venous reservoir. In one example, the extracorporeal therapy device can involve plasma exchange.

Whole blood and blood serum from mammals can be used in the present invention. The amount of blood or blood serum that can be used in the claimed methods is not intended to be limited. It can range from less than 1 mL to above 1 L, up to and including the entire blood volume of the patient when continuous recirculation back to the patient is employed. One or more ‘passes’ through the adsorption bed may be used if needed. The blood may be human or animal blood.

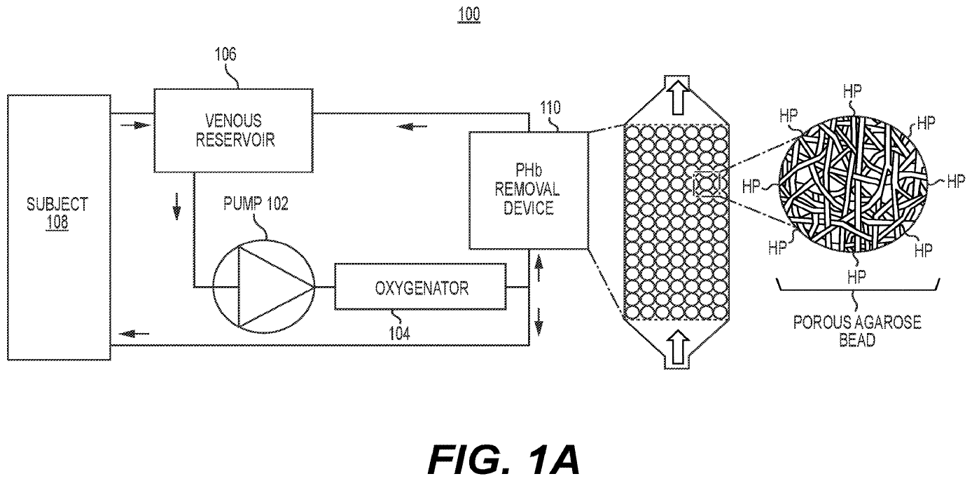

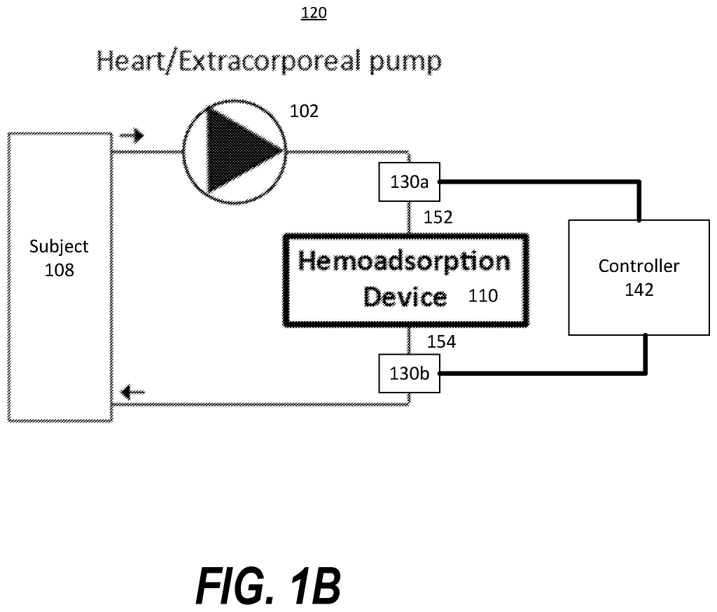

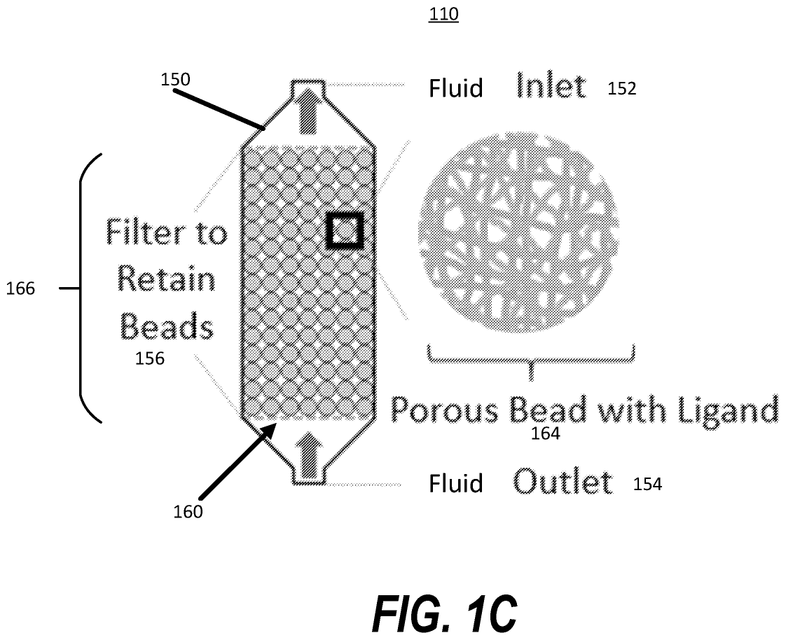

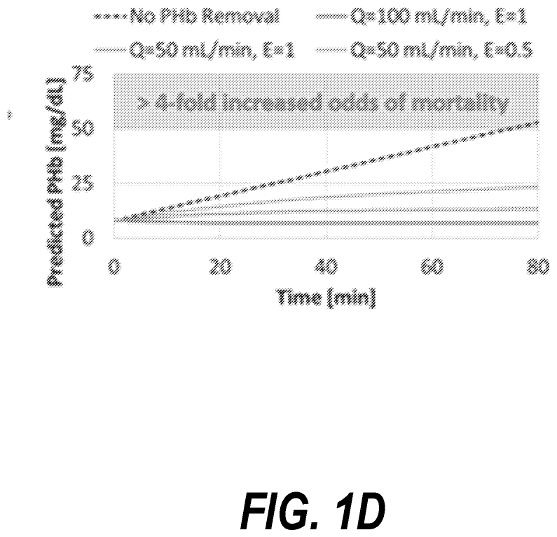

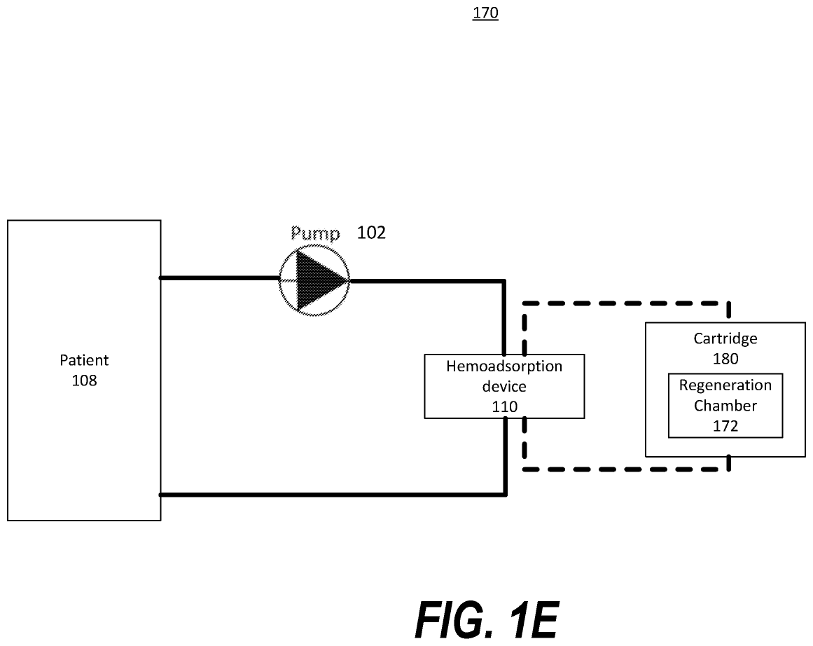

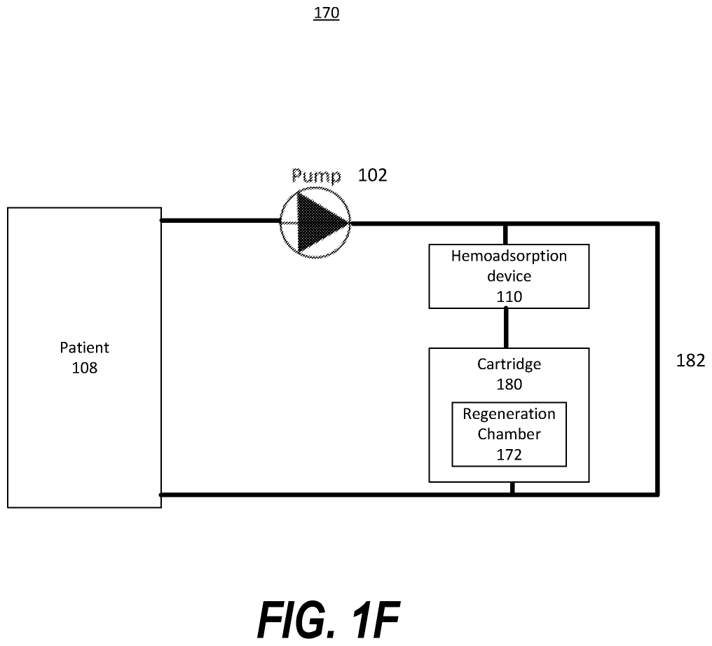

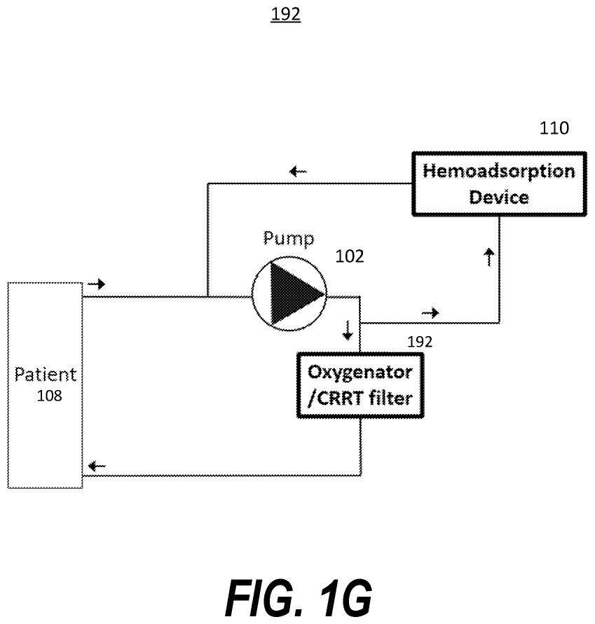

FIG. 1C illustrates the extracorporeal hemoadsorption device 110 also shown in FIG. 1A 1B, and 1E. The extracorporeal hemoadsorption device 110 can include a housing unit 150, and capture structures 164 within the housing unit 150. Optionally, the capture structures 164 are bound with ligand specific for the target component to be removed from the bodily fluid, and tubes that connect to the various components of the extracorporeal therapy system, or directly to the patient. Alternatively or additionally, the capture structure 164 can include a bead including a polymer attached to the bead. A non-limiting example of a polymer that can be included in the capture structure 164 is polyethylene glycol (PEG).

As shown in FIG. 1C, fluid can flow from the fluid outlet 154 to the blood inlet 152. Optionally, the fluid inlet 152 can be in fluid communication with the venous reservoir and/or the subject so that the fluid flows into the venous reservoir 106 and/or subject from the fluid inlet 152. As another example, the fluid can be blood, and the extracorporeal hemoadsorption device 110 can be configured so that blood enters the extracorporeal hemoadsorption device 110 at a proximal end from a blood reservoir, and so that blood exits the hemoadsorption device at a distal end and returns directly to the patient via a catheter.

The capture structure 164 for use in the devices and systems disclosed herein can be any structure capable of having a ligand conjugated on the surface thereof. As shown in FIG. 1C, the capture structures 164 can optionally be formed as one or more “beads” in the extracorporeal hemoadsorption device 110. All suitable capture structures should provide high surface area while promoting the conveyance of adsorbates to the ligand that bind them. The capture structures 164 disclosed herein can be made of materials that are sufficiently rigid to resist deformation/compaction under the encountered flow rates. Resistance to deformation is necessary to maintain the free volume and subsequent low pressure drop of the packed bed. The beads or other high-surface-area substrates may be made of biocompatible materials, such as polymers or non-polymeric material, that is essentially free of leachable impurities including glass, cellulose, cellulose acetate, chitin, chitosan, crosslinked dextran, crosslinked alanese, polyurethane, polymethylmethacrylate, polyethylene or co-polymers of ethylene and other monomers, polyethylene imine, polypropylene, polysulfone, polyacrylonitrile, silicone and polyisobutylene. Examples of useful substrates include nonporous Ultra High Molecular Weight PolyEthylene (UHMWPE). Other suitable beads are polystyrene, high density and low density polyethylene, silica, polyurethane, and chitosan. An example of the capture structure is a porous cross-linked agarose bead. While the beads are porous, the capture mechanism need not be size exclusion based. The benefit from the beads being porous is that they offer more surface area than non-porous beads. Because the capture mechanism is not based on size exclusion, there is not a threshold pore size that is needed.

When a bead is used as the capture structure 164, the bead can be any size or dimension that will promote binding of the component to be removed from blood. For example, the bead diameter can be about 10, 20, 30, 40, 50, 60, 70, 80, 90, 100, 150, 200, 250, 300, 350, 400, 450, 500, 550, 600, 650, 700, 750, 800, 850, 900, or 1000 μm. In one embodiment, the bead diameter is between 50 and 500 μm. In one particular example, the bead diameter can be about 90 μm.

The size of the channels or interstitial space between individual beads for extracorporeal blood filtration should be optimized to prevent a high-pressure drop between the inlet and outlet of the cartridge, to permit safe passage of the blood cells between the individual beads in a high flow environment, and to provide appropriate interstitial surface area for binding of the ligand to the species to be removed from the blood.

Optionally, the extracorporeal hemoadsorption device 110 is shaped as a column 166, as shown in FIG. 1C, where the captures structures 164 are “packed” in the column 166. A column 166 can be packed with any capture structures which can be beads or either woven or non-woven (sometimes heparinized) fabric. The size of the channels or space between individual beads can be adjusted by controlled packing of the beads. By controlling the fiber size of fabric, the interstitial pore size can be controlled. Non-woven fabrics are also known as felts, and have a random orientation held together by entanglement of the fibers and adhesion. Woven fabrics have a defined non-random structure. A column can be packed with fibers or yarns made from fibers. Polyethylene, and other fibers, can be drawn into thin hollow or solid fibers, that can be packed into cartridges similar to conventional hemodialysis cartridges. Additionally, these fibers can be woven into a yarn.

The ligand, which can be conjugated to the capture structure 164, can be any molecule capable of “capturing” the target, or component, to be removed from the fluid of the subject. For example, the target to be captured can be a protein, such as cell-free plasma hemoglobin. When this is the case, the ligand can be any composition capable of selectively binding the hemoglobin, such as zinc, haptoglobin, or hemoglobin-binding protein (HBP). In other examples, the protein to be removed from the subject's body fluid can be an antibody, cytokine, or toxin. It can also be a virus or bacteria. The ligand can be conjugated to the capture structure through means known to those of skill in the art. For example, the ligand can be covalently conjugated, or conjugated by adsorption, or affinity binding.

Example 1A shows a schematic of a system 100 including an extracorporeal hemoadsorption device 110 that can be used for extracorporeal therapy. In this embodiment, the fluid can be a bodily fluid, such as blood. The blood can be removed from the patient. In an optional embodiment, a pump 102 and an oxygenator 104 can be included, but these components are not necessary for some uses of the device described herein.

Optionally, the system includes a venous reservoir 106. The veinous reservoir 106 can be in fluid communication with the pump 102, extracorporeal hemoadsorption device 110 and a subject 108. The fluid can pass from the venous reservoir 106 to the extracorporeal hemoadsorption device, where unwanted components can be removed from the fluid. The fluid can then be returned to the subject 108.

Optionally, the system 100 shown in FIG. 1A can be configured without the venous reservoir 106 or the oxygenator 1404. FIG. 1B illustrates a system 120 according to an alternative embodiment of the system 100 shown in FIG. 1A. The system 120 includes a subject 108, a pump 102, and a extracorporeal hemoadsorption device 110. Optionally the pump and/or extracorporeal hemoadsorption device 110 are configured with adjustable fluid resistance to control the flow of fluid through the device.

Still with reference to FIG. 1B, optionally the system 120 can further include one or more sensors 130a, 130b. The sensors 130a, 130b can be configured to measure a property of the fluid in the system 120. Optionally, the sensors 130a, 130b can be optical sensors configured to measure the absorbance of a wavelength of light (e.g., approximately 540 nm). The absorbance of different wavelengths of light can be used to determine the composition of the fluid. As a non-limiting example, the sensors 130a, 130b can be configured to measure the concentration of hemoglobin in the fluid.

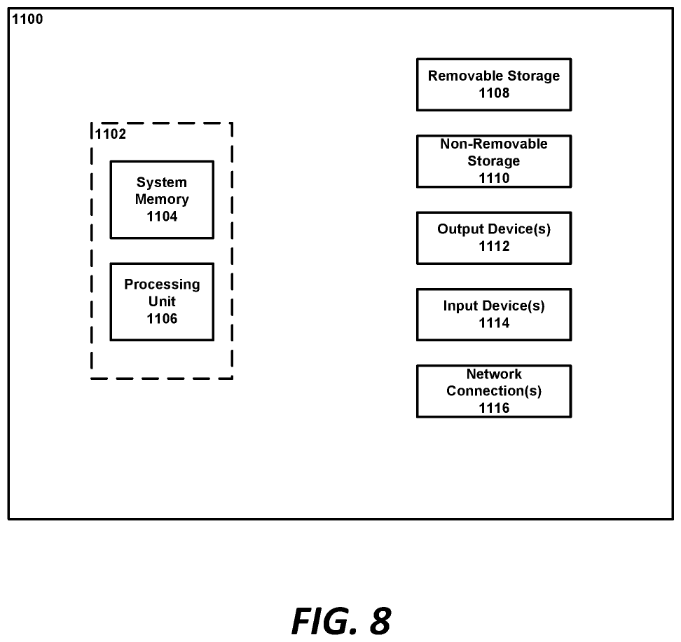

In the system 120 shown in FIG. 1B, the hemoadsorption device 110 includes a fluid inlet 152 and a fluid outlet 154. The sensor 130a can measure a first concentration of an impurity in the fluid at the fluid inlet 152. The sensor 130b can measure a second concentration of an impurity in the fluid at the fluid outlet 154. By comparing the concentrations measured at 130a and 130b, the effectiveness of the hemoadsorption device 110 can be determined. Optionally, the system 120 can include a controller 142 operably coupled to the sensors 130a, 130b. The controller 142 can include a computing device (e.g., the computing device 1100 shown in FIG. 8). The controller 142 can determine an amount of impurities in the fluid after the blood passes through the hemoadsorption device. Optionally, the amount of impurities in the blood can be used to determine the effectiveness of the fluid adsorption device. Optionally, the controller 142 can display sensor information, for example using a display, or any of the output devices 1112 described with reference to FIG. 8. Alternatively or additionally, the controller can determine when the hemoadsorption device should be recharged or replaced based on the outputs of the sensors 130a, 130b, and optionally display to the user when the hemoadsorption device should be recharged or replaced. It should be understood that the two sensors 130a, 130b described in FIG. 1B are intended only as a non-limiting example, and that implementations of the present disclosure can include any number of sensors, or different combinations of sensors. Embodiments of the present disclosure can optionally include integrated flow sensors and/or integrated pressure differential sensor.

An example embodiment includes systems, devices, and methods of obtaining continuous, live measurements of the concentration of Hb in plasma are disclosed herein. Along the extracorporeal circuit can be placed a Y-connector with one arm containing an inline plasma filter flush with the non-filtered pathway. The filtered stream can be pumped through a spectrophotometric sensor capable of measuring the optical presence of Hb in the plasma. The sensor can optionally measure multiple wavelengths at once, producing a continuous signal. This can be interpreted by a computer (e.g., the computing device 1100 illustrated in FIG. 8) to display a current reading of the concentration of free Hb. The filtered stream would reconnect to the unfiltered stream and continue on the circuit. Optionally, the Y-connector, hemoadsorption device, and spectrophotometric sensor can be placed on either side of the hemoadsorption device, allowing precise measurement of the reduction of free Hb.

Simulations of the example embodiments described herein show that embodiments of the present disclosure can prevent PHb levels from rising in a simulation of pediatric CPB. FIG. 1D illustrates an example of predicted PHb during pediatric CPB when blood is filtered at different rates.

In some embodiments of the present disclosure, the extracorporeal hemoadsorption device 110 can be configured so that it can be recharged after a period of use. For example, with reference to the system 170 illustrated in FIG. 1E, a regeneration chamber 172 can be used to recharge the extracorporeal hemoadsorption device 110. In the system 170, the regeneration chamber 172 is part of a cartridge 180. The cartridge 180 can be in fluid communication with the extracorporeal hemoadsorption device 110 so that a reagent in the regeneration chamber 172 can flow into the extracorporeal hemoadsorption device 110. Optionally, the system 170 can be configured so that the extracorporeal hemoadsorption device 110 is detachable from the system 170 and can be attached to the cartridge 180 to circulate fluid between the cartridge 180 and the extracorporeal hemoadsorption device 110. In some embodiments of the present disclosure, the reagent in the cartridge 180 includes guanidine and the regeneration chamber 172 can be configured to circulate guanidine through the extracorporeal hemoadsorption device.

In some embodiments, the cartridge 180 can be placed “in line” with the extracorporeal hemoadsorption device 110, as shown in FIG. 1F. It should be understood that the cartridge 180, pump 102, and extracorporeal hemoadsorption device 110 can be connected in different orders using different combinations of catheters or conduits, and that the configurations of cartridge 180, extracorporeal hemoadsorption device 110, and pump 102 illustrated in FIGS. 1E and 1F are intended only as non-limiting examples. As shown in FIG. 1F, the system can optionally include a bypass conduit 182 configured to allow fluid flow past the hemoadsorption device 110. With reference to FIG. 1G, a system 190 is shown with an oxygenator and continuous Renal Replacement Therapy (CRRT) filter 192. The system 190 can include the pump 102 configured to move fluid through both the CRRT filter 192 and the hemoadsorption device 110, before the blood is recirculated to the patient 108.

When a pump or fluid-moving device is used with the invention, various pressure arrangements are possible. In certain aspects, it may also be desirable to switch from one pressure range to another pressure range and back and forth, or from a series of pressure ranges, For example, it may be desire in certain treatments to move from a high pressure to low pressure range and sometimes back and forth between pressure ranges. By low pressure we mean ranges of from 70 mmHg to 120 mmHg, 85 mmHg to 130 mmHg, 85 mmHg to 110 mmHg, or 90 mmHg to 120 mmHg as measured at by what is delivered to the body. By high pressures we mean pressure from 200 mmHg to 500 mmHg, 200 mmHg to 400 mmHg, 200 mmHg to 300 mmHg, 220 mmHg to 350 mmHg, or 250 mmHg to 340 mmHg as delivered to the body. It is also possible to delivery other pressure ranges of 70 mmHg to 500 mmHg, 110 mmHg to 250 mmHg, 120 mmHg to 200 mmHg, or 120 mmHg to 160 mmHg.

It should be understood that, the pump 102 illustrated in FIGS. 1A, 1B, 1E and 1F can include any pressure source or pump(s) that can move fluid through the extracorporeal hemoadsorption device 110. Examples of pumps that may be used include rotary pumps, roller pumps, pulsating pumps, non-pulsating pumps or combinations thereof. In addition, the above treatment pressure can be combined with the flow volumes of from 10 ml to 1400 ml per minute, 5 ml to 40 ml, per minute, 10 ml to 25 ml per minute, 25 ml to 1000 ml per minute, 50 ml to 1200 ml per minute, 10 ml to 180 ml per minute, 100 ml to 250 ml per minute, 140 ml to 500 ml per minute, 100 ml to 800 ml per minute, 500 ml to 1400 ml per minute. The desired treatment pressures and flow volumes will depend on the treatment being performed.

In certain aspects, it may be desirable to use or add equipment to filter the blood or fluids being circulated in the treatment system. In certain aspects, it may be desirable to use or add blood oxygenators, such as membrane or bubble oxygenators, hyperthermic treatment equipment, hypothermic equipment, dialysis equipment, devices that permit the taking of artery or vein blood samples, monitoring equipment, filtering equipment, balloon catheter monitoring and control devices such as counter pulsation devices for cardiac applications or balloon pressurization controllers, (for example, for cardiac application every time heart beats the balloon may be deflated and every time the heart rests the balloon may be inflated), computer systems for controlling or monitoring the various equipment, external tubing, flow controllers, drug delivery devices, blood monitoring devices, such as blood pH, SO2, pulse or other blood monitoring devices, sampling devices, nutrient suppliers (such as saline or dextrose drips), blood or fluid cleaning or scrubbing devices (including, for example, chemical and physical filters), blood temperature control devices or other suitable devices or combinations thereof. In certain aspects, it may be desirable to add or use devices or equipment where the fluid may be monitored and/or sampled, may have its chemical, physical or kinetic properties modified and may have various substances added to and/or removed from it in accordance with a specific treatment regimen and according to the individual device or devices used.

Another optional embodiment includes arrangements such that disposable elements within the device can be replaced during use. For example, in one such arrangement the device could contain multiple bead-containing cartridges that are arranged in parallel and cartridges could be replaced as the ligands on the beads within the cartridge become saturated with the target species. The bead cartridges/columns could be removed and replaced from a more permanent housing without stopping blood flow through the device. The permanent housing of the device can optionally have features that include an integrated flow sensor, which can include detection of bubbles or air entrainment. It can also include an integrated pressure differential sensor, which can give information about clotting. Further, it can include an integrated mechanism to adjust resistance that can allow modification of blood flow through the device.

Built-in sensors within the device can be used to measure cell-free hemoglobin for various purposes including overall systemic levels of cell-free plasma hemoglobin as well as saturation of the disposable device requiring a cartridge change. These sensors can either detect or calculate the saturation of the bead column with cell-free plasma hemoglobin. Also disclosed are sensors that allow continuous measurement of cell-free hemoglobin levels in various areas of the filter.

Also disclosed herein is a method for removing a target component from a subject, the method comprising: removing blood from a subject; and exposing said blood from step a) to a purification column, wherein said purification column comprises at least one capture structure, wherein a ligand is conjugated on a surface of said capture structure, wherein said ligand is specific for a component to be removed from the blood of the subject, thereby removing said component from the subject's blood; and returning the blood to the subject.

This removal of unwanted components can be done over a selected time period and can be repeated as treatment dictates using the ability of the access device to provide intermittent and recurrent access to the arterial and/or venous circulation of the patient. In certain aspects, the removal of unwanted components can be carried out for a period of time and then stopped for a period of time. The desired treatment can dictate the number and lengths of the periods of removal of unwanted components as well as the periods of rest between treatments.

The systems, methods and devices herein can be used with a number of other components. For example, the subject can be treated with drugs or drug solutions, anticoagulants, antibiotics, contrast fluids, diagnostic fluids, therapeutics, nutrients, saline, buffers, plasma, synthetic or natural blood products or factors, antibodies, proteins or fragments thereof, peptides or fragments thereof, genes or fragments thereof, DNA, RNA, nucleic acids, nano devices, blood cells and/or combinations of one or more of the above.

### EXAMPLES

### Example 1: Extracorporeal Hemoadsorption Device

Disclosed herein is an extracorporeal hemoadsorption device that represents an easily implementable therapy for PHb removal during ECT. This is based on the facts that PHb has detrimental bioactive effects with a role in ECT-associated AKI and that Hp represents an ideal ligand with high selectivity for PHb. This device can be applied to any form of ECT where PHb is produced and can make a substantial positive impact on patient outcomes.

The device disclosed herein can be a hemoadsorption device comprising a column containing porous agarose beads (mean diameter −90 μm) with Hp covalently bound to the bead surface. Similar to the operation of an affinity column, as blood flows through the bead-based matrix, PHb removal occurs via its irreversible binding to bead-bound Hp. The device is intended to be implemented within an extracorporeal support circuit as shown in FIG. 1A which enables blood flow through the device without the need for an additional pump and diverts only a minor portion of total pump flow. This type of configuration was shown to be safe and feasible during clinical trials of the Cytosorb® extracorporeal cytokine adsorber⁴⁷. A lumped parameter model was used to predict the ability of varying device flow rates and other system parameters to achieve therapeutic levels of PHb removal. The model considers the patient and support circuit as a single well-mixed reservoir with a constant PHb generation rate due to ongoing hemolysis. PHb removal efficiency by the device is described by E which is defined as the fraction of PHb removed from blood passing through the device and ranges from 0 (no PHb removal) to 1 (complete PHb removal). Preliminary modeling used published patient and PHb data from a large cohort of pediatric CPB patients (mean CPB duration=78 min)²⁰. Predicted patient PHb levels for varying device flow rates (Q) and E values are shown in FIG. 1B. The dotted line in FIG. 1B represents the baseline case of no PHb removal and corresponds to published clinical data²⁰. As shown, Q=100 mL/min and E=1 results in an 8-fold reduction in PHb at the conclusion of CPB vs. baseline. Even at Q=50 mL/min and E=0.5, PHb is maintained well below the threshold associated with increased mortality⁴³⁷. The modeled device flow rates correspond to only 4-7% of overall CPB flow rate (assuming CPB flow rate=110 mL/kg/min) and less than 30% of the device flow rates that were used in the Cytosorb® clinical trial of CPB⁴⁷. This model shows that the device flow rates required for therapeutic PHb removal are feasible during ECT.

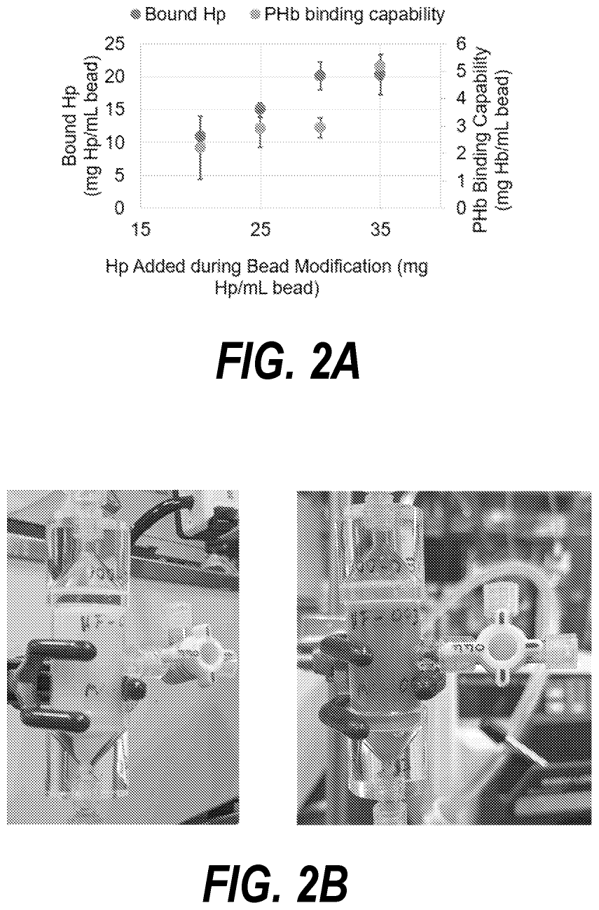

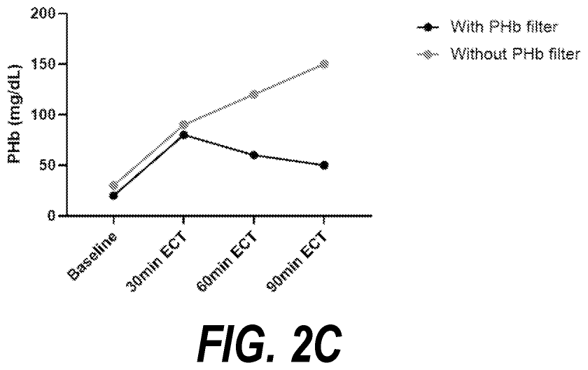

Methods of producing Hp-modified beads and their ability to bind PHb are demonstrated herein. Native human Hp (polymorphic mixture of 1-1, 2-1, and 2-2 phenotypes; Abcam, MA) was immobilized on agarose beads (Cytiva, MA) via stable amide bonds between N-hydroxy-succinimide and primary amino groups on the beads and Hp, respectively. Additionally, Hp was conjugated to low MW polyethylene glycol via stable amide bonds between N-hydroxy-succinimide and primary amino groups on the polyethylene glycol and Hp, respectively, and then the modified protein was immobilized on agarose beads (Click Chemistry Works, AZ) creating a stable 1,2,3-triazole conjugation Modified beads were assessed for their ability to bind PHb during agitated incubation with plasma from hemolyzed rat blood. FIG. 2A shows the PHb binding capacity of beads loaded with varying levels of Hp. Beads were loaded with up to 20.3 mg Hp/mL bead which exhibited a corresponding PHb binding capacity of 5.2 mg PHb/mL bead. FIG. 2B illustrates that Hp-modified beads exhibited clear red color change due to PHb binding. Assuming an average Hp MW of −229 kDa (assumes equal portions of each phenotype) and 1:1 Hp-PHb binding, an expected theoretical binding capacity would be 0.3 mg PHb/mg Hp. This expected value matches that exhibited during in vitro experiments, demonstrating that immobilized human Hp not only retains its functional activity but can also bind rat PHb. Similar levels of binding were also observed in solutions of lyophilized human Hb in saline. For the PHb binding capacity shown in preliminary data, total PHb removal for the case with the largest PHb removal rate modeled in FIG. 1B (blue line) corresponds to a total bead volume of <80 mL. Total protein levels in rat plasma following incubation with Hp-modified beads were within 5% of pre-exposure values, showing low levels of non-specific protein removal.

A rat ECT model investigated the effect of the device on plasma hemoglobin levels. An adult rat was put on a ECT circuit with a pump, oxygenator, reservoir, and the device according to an embodiment of the present disclosure. Blood samples were taken at baseline, 30, 60, and 90 minutes and measured using the HemoCue Low Hb/Plasma system. The data was compared to the same model of an adult rat with no device present. The study (n=1 in each group) with embodiment of the present disclosure demonstrated significant reduction in pHb levels as illustrated in FIG. 2C. FIG. 2C represents a comparison of PHb levels with and without the PHb filter according to an embodiment of the present disclosure.

Methods of removing Hb from the Hb-Hp complexes formed under usage of the device are demonstrated herein. A column containing bead-bound Hb-Hp complexes was exposed to 5 M guanidine hydrochloride (aq.) at 5 mL/min for 100 minutes. The column was reassessed and demonstrated 57% of the original Hb capture ability. Similar regeneration washes can be used such as urea, magnesium chloride, and other elutes that disrupt ionic interactions.

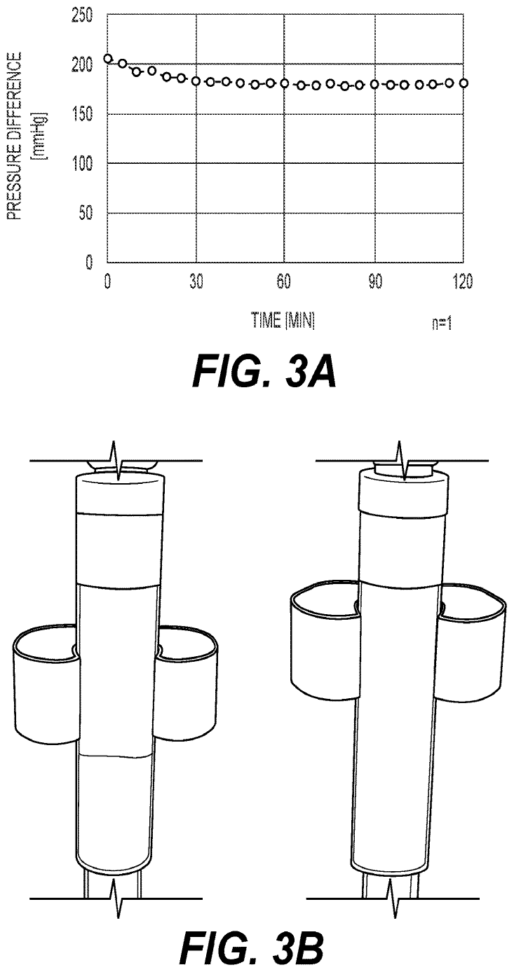

Studies assessing the feasibility of whole blood flow through bead columns have also been performed. Plastic column housings contained −5 mL of non-functionalized agarose beads (no immobilized Hp). Manifolds at the column ends contained a 40-μm polyester screen (taken from SQ40 Microaggregate Blood Transfusion Filters; Haemonetics, MA) to allow for the passage of blood while retaining the beads. Purchased rat whole blood (Lampire Biological Laboratories, PA) anticoagulated with heparin and citrate was recirculated at 2 mL/min through a column for 2 h while measuring the trans-column pressure difference. Pressure difference remained relatively constant throughout recirculation (FIG. 3A) thereby indicating unobstructed blood flow and a lack of any significant changes to the bead bed. Uniform filling of the column during priming (FIG. 3B) indicated relatively well-distributed blood flow. The rinsed column following recirculation (FIG. 3B) also showed the absence of any loss of beads or significant accumulation of any particulates or clots. These results demonstrate the feasibility of whole blood flow through columns of agarose beads.

1. Benchtop Studies of Scaled-Down Columns Under Whole Blood Flow Characterize the Dependence of E on the Critical Operating Parameters of Column Flow Rate and Bead Saturation with PHb.

### Methods:

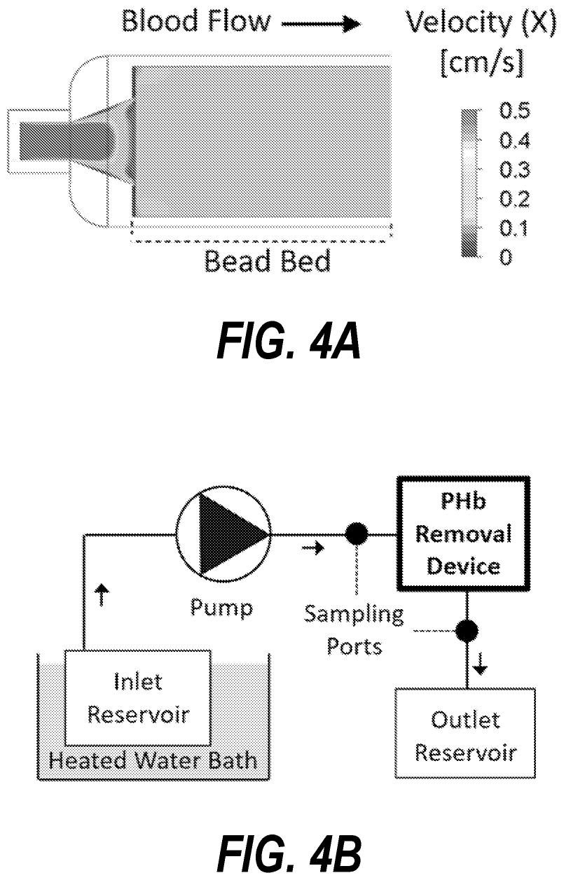

Prototype design and benchtop setup: Prototypes similar to those used in preliminary testing (FIG. 3B) are fabricated with a column height and diameter of 6.5 and 1.2 cm, respectively, resulting in a total bead volume of ~7.5 mL. This bead volume is based on data of PHb bead binding capacity and targeted levels of PHb removal during rat studies. Hp used for prototype fabrication is provided by CSL Behring through a collaborative research agreement. Beads are modified with sterile reagents and introduced into pre-sterilized housings. Computational fluid dynamic (CFD) modeling (SolidWorks Flow Simulation 2020; Dassault Systemes, MA) was used to predict blood velocity and pressure in the proposed design. Blood was modeled as a non-Newtonian fluid and the packed bed of beads as a continuous porous media with a permeability specified according to the Ergun equation. Results (FIG. 4A) predict an adequately low resistance (~40 mmHg/mL/min) and uniform flow distribution through the bead bed for the maximum blood flow rate anticipated during rat trials (4 mL/min). Spatially uniform flow is critical to efficient PHb removal and avoidance of thrombosis.

PHb binding is assessed during single-pass flow in rat whole blood using the benchtop setup shown in FIG. 4B. Anticoagulated blood is collected from rats. A small volume of blood is hemolyzed via freezing and thawing and then centrifuged to collect concentrated PHb. PHb is added to whole blood to achieve a concentration of 170 mg/dL, corresponding to the median peak PHb from rat ECT data. PHb-adjusted blood is pumped from an inlet reservoir through the column using a peristaltic pump. Blood samples (~1 mL) are collected at the device outlet every 5 minutes and centrifuged to collect plasma for PHb measurement. Inlet conditions can be constant due to single-pass flow; however, inlet samples are assessed periodically to ensure consistency. Experimentation continues until there is no measurable difference between inlet and outlet PHb concentrations (i.e., no measurable PHb removal), thereby indicating complete PHb bead saturation.

Measurements and Calculated Parameters: PHb is quantified using either a Hemocue® photometer or well-based assay based on the improved Triton/NaOH method. PHb removal efficiency (E) is calculated via Eq. 1. Trials are repeated in triplicate at blood flow rates of 1, 2, and 4 mL/min. These flow rates were selected based on targeted PHb removal during rat trials and preliminary modeling. Cumulative PHb mass bound to the column (mpHb) and column saturation (S) is calculated for each time point via Eqs. 2 and 3, respectively. E is expected to decrease towards zero as the column approaches complete saturation. Regression analysis is used to develop empirical relationships between E and S at the varying flow rates. These relationships can be used with the theoretical model to predict circulating PHb during device implementation and guide operating parameters during subsequent in vivo testing.

$$PHb Removal Efficiency ( E ) = 1 - \frac{{C}_{outlet}}{{C}_{inlet}} \qquad Eq . 1$$

$${C}_{outlet} : PHb conc at column outlet$$

$${C}_{inlet} : PHb conc at column inlet$$

$$Cumulative PHb Mass Bound to Column ( {m}_{PHb} ) = {\int }_{0}^{t} {QC}_{inlet} E dt \qquad Eq . 2$$

$$Column Saturation ( S ) = \frac{{m}_{PHb}}{B} \qquad Eq . 3$$

$$B : column PHb binding capacity ( based on loaded Hp )$$

### 2. Assess the Feasibility and Therapeutic Efficacy of the Prototype Devices In Vivo in a Previously Developed Rodent Model of ECT.

A rat ECT model that parallels the biochemical changes seen in humans allows the assessment of the feasibility and efficacy of a device prototype to remove PHb and ameliorate

### AKI.

Methods:

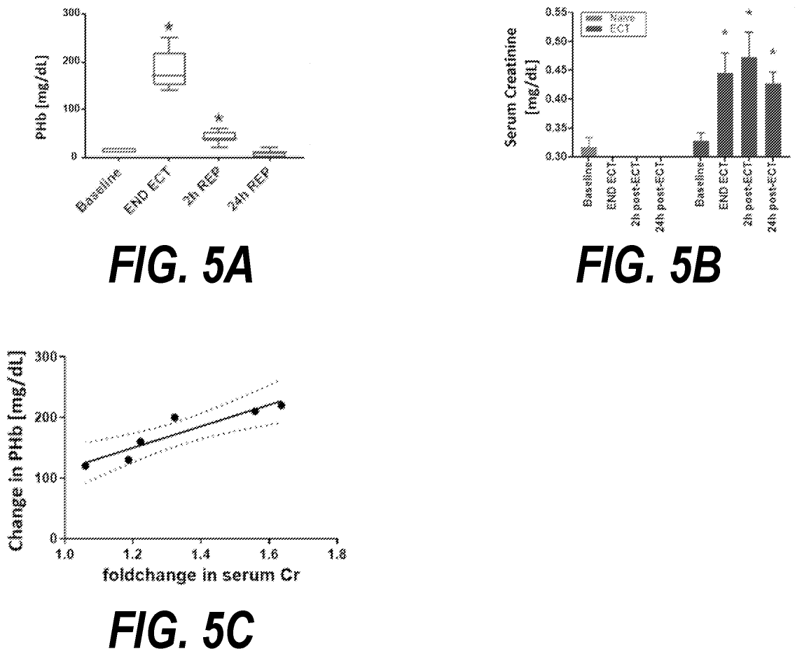

Experimental rat model of ECT: Studies use an established experimental model of rat ECT at the Safar Center for Resuscitation Research. Preliminary rat ECT studies show temporal PHb levels that parallel human clinical data²⁰ where PHb levels rise at the end of 60 min of ECT and return to baseline by 24 h post-ECT (FIG. 5A). Serum creatinine (SCr) levels increase by the end of ECT and remain elevated (FIG. 5B) and their change is correlated with the change in PHb (R²=0.86, p<.01) (FIG. 5C).

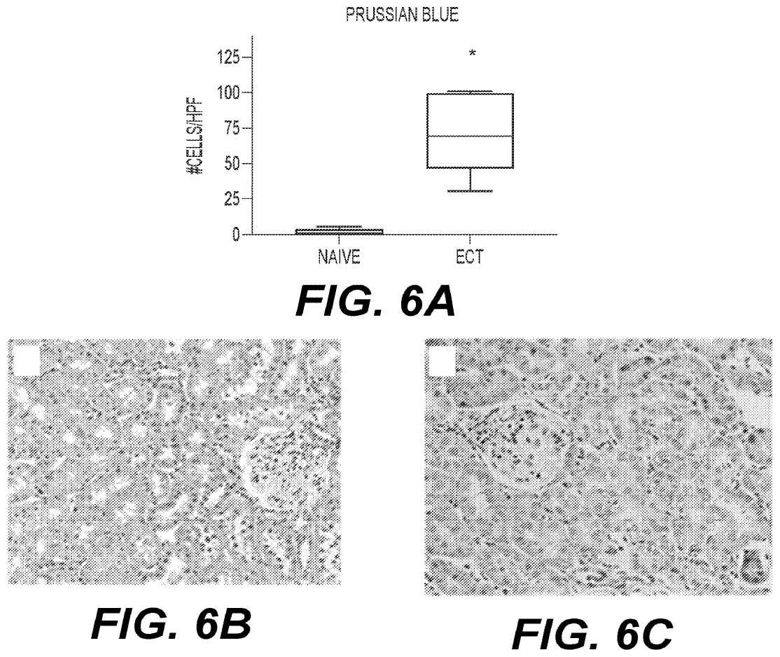

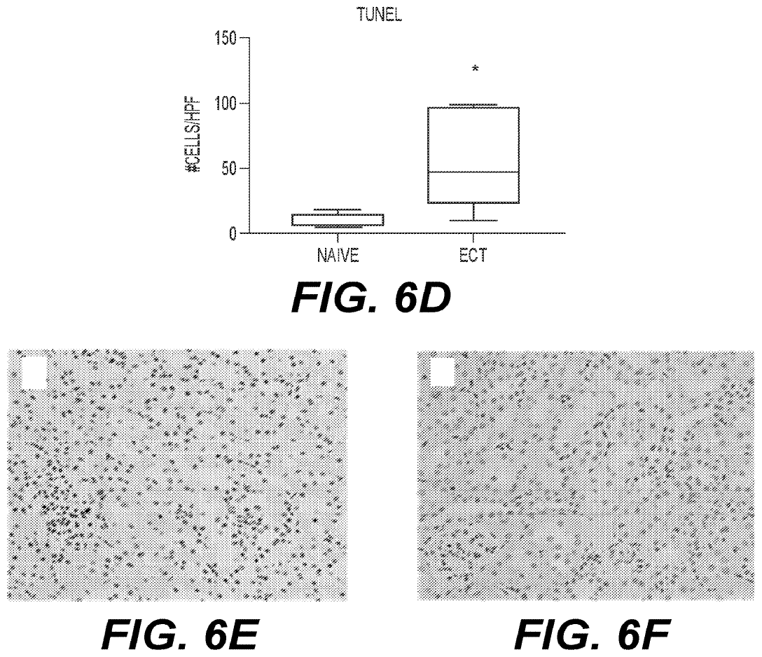

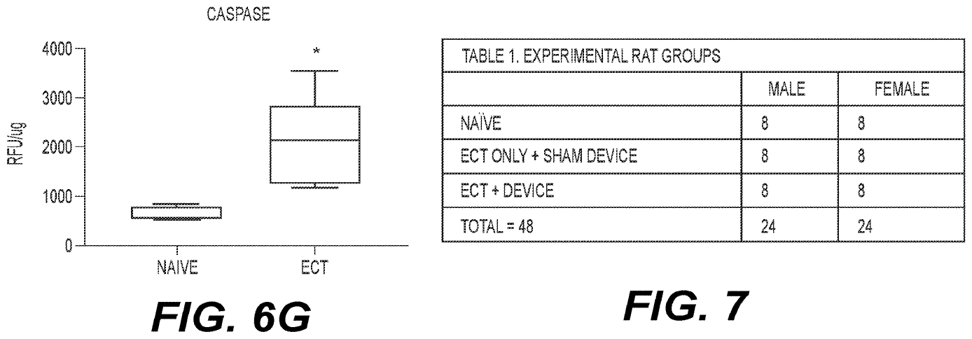

Kidney sections from rats undergoing ECT showed increases in Prussian blue hemosiderin staining in the renal tubules (FIG. 6A-C), TUNEL staining (FIG. 6D-F), and caspase activity (FIG. 6G) which are consistent with hemosiderin deposition and apoptotic cell death.

Male and female Sprague-Dawley rats (~400 g) are anesthetized with 4% isoflurane in FiO₂ 1.0, intubated, and mechanically ventilated using a piston ventilator (Harvard rodent apparatus). Anesthesia is maintained with 1-4% isoflurane in FiO₂ 0.5. The left femoral artery and vein are cannulated for blood sampling, continuous blood pressure and heart rate monitoring, and medication/fluid administration. Mean arterial blood pressure, electrocardiogram, and rectal and tympanic temperatures are monitored continuously. The ECT circuit has been described previously⁵⁴ and consists of a custom-designed oxygenator, open reservoir, and roller pump which is primed with fresh donor blood. The right femoral artery and right jugular vein are cannulated for ECT and all rats are subjected to 60 min of ECT at 60 mL/min. Heparin is used to maintain ACT >400 s. Derangements of electrolytes and acid-base status is corrected with calcium chloride, sodium bicarbonate, and/or ventilator adjustments.

Rats are assigned randomly to treatment groups illustrated in FIG. 7, which consists of ECT with a sham device (column containing non-functionalized beads) and ECT with the device containing Hp-modified beads. The devices is incorporated into the ECT circuit as shown in FIG. 1A. To address rigor, the surgical research technician is blinded, and the PI assesses the outcomes in a blinded fashion. Blood flow rate through the column is modified via an adjustable Hoffman clamp inserted post-column. Device blood flow rate is adjusted to target maintenance of PHb <50 mg/dL and is guided by the results and modeling from above. After 60 nun, rats are weaned from ECT and isoflurane anesthesia continues for 2 hours post-ECT. Blood and urine are collected at baseline, End ECT, and 2 hours post-ECT. After 2 hours, catheters and probes are removed, a Mini-mitter probe is placed in the peritoneal cavity for post-operative monitoring and temperature control (37° C.), and rats are placed individually in a cage with free access to food, water, and a 12 hour light/dark cycle until 24 hours post-ECT when final blood and urine samples are collected and organs are harvested.

Assays: Serum and urine Cr levels are measured by Kansas State Veterinary Laboratory. Serum cystatin C and urine NGAL and KIM-1 are measured via ELISA. PHb levels are measured via the Hemocue® photometer. A complete blood count is performed at baseline and 24 hours post-ECT. In order to confirm specificity of the device, beads from used devices are washed and stripped after experiment completion and a proteomic analysis of the effluent is completed using the mass spectrometry service at the Biomedical Mass Spectrometry Center at the University of Pittsburgh.

Histology/Immunohistochemistry: Cells are counted in a blinded fashion and averaged as the #positive cells/high powered field (hpf) with each kidney section divided into 6 hpfs. Stains include: Prussian blue staining for hemosiderin deposition; periodic acid schiff stain for the structure of the renal glomeruli, tubules, and the basement membrane; and TUNEL staining to evaluate cell-death. Immunohistochemistry is used to evaluate the expression of KIM-1 and F4/80 for tubular injury and inflammation, respectively.

Statistical analysis: In order to account for the repeated measures and clustering of data within subjects across the four time points, time-course analysis with general linear models is used, and the longitudinal data modules of STATA (xt) are also used to examine differences in levels over time.

Based on the observed average peak SCr (0.52 mg/dL±0.17) in studies on ECT in the model and with the rigorous goal of preventing an increase in SCr above baseline (0.32 mg/dL±0.04), a sample size of 8 per group detects this difference with α=0.05 and β=0.20 (FIG. 7).

### Example 2: Extracorporeal Hemoadsorption Device

Extracorporeal therapies (ECT) including cardiopulmonary bypass (CPB) and extracorporeal membrane oxygenation (ECMO) have been refined over the years, yet unfavorable outcomes such as acute kidney injury (AKI) continue to occur and are associated with mortality and prolonged intensive care unit and hospital length of stay. Increased cell-free plasma hemoglobin (PHb) from hemolysis during CPB and ECMO has been identified as playing a central role in such dysfunction and is thus an obvious clinical target. Despite this, there are no clinically available therapies for the selective removal of PHb during ECT. Disclosed herein is a novel extracorporeal hemoadsorption device to serve as an easily implementable therapy to selectively remove PHb during ECT. The hemoadsorption device can be a bead-based matrix containing immobilized haptoglobin (Hp), a native protein with high-affinity binding for PHb, to selectively remove PHb from whole blood flow.

Disclosed herein are methods to produce Hp-modified beads, confirmation of the ability of these beads to bind PHb, demonstration of the ability of a bead-based column to accommodate whole blood flow, and model-based evidence of the ability for clinically reasonable device flow rates to achieve therapeutic PHb removal. In addition, a rat model of ECT-induced AKI has been developed to be used during in vivo assessment.

Steps include (i) fabricating functional, scaled-down prototypes and characterize PHb binding kinetics during benchtop studies in whole blood flow and (ii) using a rat model to evaluate the feasibility of device implementation during ECT and its ability to prevent ECT-induced renal injury. Thus, the disclosed work involves elements of design and fabrication of extracorporeal blood-contacting devices, porous media fluid dynamics, blood-based mass transfer, hemocompatibility, kidney injury, hemolysis-associated pathology, and clinical implementation of ECT.

It should be appreciated that the logical operations described herein with respect to the various figures may be implemented (1) as a sequence of computer implemented acts or program modules (i.e., software) running on a computing device (e.g., the computing device described in FIG. 8), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 8, an example computing device 1100 upon which the methods described herein may be implemented is illustrated. It should be understood that the example computing device 1100 is only one example of a suitable computing environment upon which the methods described herein may be implemented. Optionally, the computing device 1100 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 1100 typically includes at least one processing unit 1106 and system memory 1104. Depending on the exact configuration and type of computing device, system memory 1104 may be volatile (such as random access memory (RAM)), non-volatile (such as read-only memory (ROM), flash memory, etc.), or some combination of the two. This most basic configuration is illustrated in FIG. 8 by dashed line 1102. The processing unit 1106 may be a standard programmable processor that performs arithmetic and logic operations necessary for operation of the computing device 1100. The computing device 1100 may also include a bus or other communication mechanism for communicating information among various components of the computing device 1100.

Computing device 1100 may have additional features/functionality. For example, computing device 1100 may include additional storage such as removable storage 1108 and non-removable storage 1110 including, but not limited to, magnetic or optical disks or tapes. Computing device 1100 may also contain network connection(s) 1116 that allow the device to communicate with other devices. Computing device 1100 may also have input device(s) 1114 such as a keyboard, mouse, touch screen, etc. Output device(s) 1112 such as a display, speakers, printer, etc. may also be included. The additional devices may be connected to the bus in order to facilitate communication of data among the components of the computing device 1100. All these devices are well known in the art and need not be discussed at length here.

The processing unit 1106 may be configured to execute program code encoded in tangible, computer-readable media. Tangible, computer-readable media refers to any media that is capable of providing data that causes the computing device 1100 (i.e., a machine) to operate in a particular fashion. Various computer-readable media may be utilized to provide instructions to the processing unit 1106 for execution. Example tangible, computer-readable media may include, but is not limited to, volatile media, non-volatile media, removable media and non-removable media implemented in any method or technology for storage of information such as computer readable instructions, data structures, program modules or other data. System memory 1104, removable storage 1108, and non-removable storage 1110 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 1106 may execute program code stored in the system memory 1104. For example, the bus may carry data to the system memory 1104, from which the processing unit 1106 receives and executes instructions. The data received by the system memory 1104 may optionally be stored on the removable storage 1108 or the non-removable storage 1110 before or after execution by the processing unit 1106.

The compositions, devices, systems, and methods of the appended claims are not limited in scope by the specific compositions, devices, systems, and methods described herein, which are intended as illustrations of a few aspects of the claims. Any compositions, devices, systems, and methods that are functionally equivalent are intended to fall within the scope of the claims. Various modifications of the compositions, devices, systems, and methods in addition to those shown and described herein are intended to fall within the scope of the appended claims. Further, while only certain representative compositions, devices, systems, and method steps disclosed herein are specifically described, other combinations of the compositions, devices, systems, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

Unless defined otherwise, all technical and scientific terms used herein have the same meanings as commonly understood by one of skill in the art to which the disclosed invention belongs. Publications cited herein and the materials for which they are cited are specifically incorporated by reference.

### REFERENCES

1. Aydin S I, Seiden H S, Blaufox A D, Parnell V A, Choudhury T, Punnoose A, Schneider J. Acute kidney injury after surgery for congenital heart disease. Ann Thorac Surg. 2012 Nov;94(5):1589-1595. PMID: 22884599
2. Blinder J J, Goldstein S L, Lee V-V, Baycroft A, Fraser C D, Nelson D, Jefferies J L. Congenital heart surgery in infants: effects of acute kidney injury on outcomes. J Thorac Cardiovasc Surg. 2012 February;143(2):368-374. PMID: 21798562
3. Mamikonian L S, Mamo L B, Smith P B, Koo J, Lodge A J, Turi J L. Cardiopulmonary bypass is associated with hemolysis and acute kidney injury in neonates, infants, and children*. Pediatr Crit Care Med. 2014 March;15(3):e111-119. PMCID: PMC3951557
4. Borasino S, Kalra Y, Elam A R, Carlisle O'Meara L, Timpa J G, Goldberg K G, Collins Gaddis J L, Alten J A. Impact of Hemolysis on Acute Kidney Injury and Mortality in Children Supported with Cardiac Extracorporeal Membrane Oxygenation. J Extra Corpor Technol. 2018 December; 50(4):217-224. PMCID: PMC6296454
5. Thongprayoon C, Cheungpasitporn W, Lertjitbanjong P, Aeddula N R, Bathini T, Watthanasuntorn K, Srivali N, Mao M A, Kashani K. Incidence and Impact of Acute Kidney Injury in Patients Receiving Extracorporeal Membrane Oxygenation: A Meta-Analysis. J Clin Med. 2019 Jul. 5; 8(7). PMCID: PMC6678289
6. Fleming G M, Sahay R, Zappitelli M, King E, Askenazi D J, Bridges B C, Paden M L, Selewski D T, Cooper D S. The Incidence of Acute Kidney Injury and Its Effect on Neonatal and Pediatric Extracorporeal Membrane Oxygenation Outcomes: A Multicenter Report From the Kidney Intervention During Extracorporeal Membrane Oxygenation Study Group. Pediatr Crit Care Med. 2016 December; 17(12):1157-1169. PMCID: PMC5138084
7. Pickering J W, James M T, Palmer S C. Acute kidney injury and prognosis after cardiopulmonary bypass: a meta-analysis of cohort studies. Am J Kidney Dis. 2015 Feb;65(2):283-293. PMID: 25445101
8. Askenazi D J, Ambalavanan N, Hamilton K, Cutter G, Laney D, Kaslow R, Georgeson K, Barnhart D C, Dimmitt R A. Acute kidney injury and renal replacement therapy independently predict mortality in neonatal and pediatric noncardiac patients on extracorporeal membrane oxygenation. Pediatr Crit Care Med. 2011 January;12(1):el-6. PMID: 20351617
9. Basu R K, Andrews A, Krawczeski C, Manning P, Wheeler D S, Goldstein S L. Acute kidney injury based on corrected serum creatinine is associated with increased morbidity in children following the arterial switch operation. Pediatr Crit Care Med. 2013 Jun;14(5):e218-224. PMID: 23439467
10. Schneider J, Khemani R, Grushkin C, Bart R. Serum creatinine as stratified in the RIFLE score for acute kidney injury is associated with mortality and length of stay for children in the pediatric intensive care unit. Crit Care Med. 2010 March;38(3):933-939. PMID: 20124891
11. Fortenberry J D, Paden M L, Goldstein S L. Acute kidney injury in children: an update on diagnosis and treatment. Pediatr Clin North Am. 2013 June;60(3):669-688. PMID: 23639662
12. Alkandari O, Eddington K A, Hyder A, Gauvin F, Ducruet T, Gottesman R, Phan V, Zappitelli M. Acute kidney injury is an independent risk factor for pediatric intensive care unit mortality, longer length of stay and prolonged mechanical ventilation in critically ill children: a two-center retrospective cohort study. Crit Care. 2011 Jun. 10; 15(3):R146. PMCID: PMC3219018
13. Bennett M, Dent C L, Ma Q, Dastrala S, Grenier F, Workman R, Syed H, Ali S, Barasch J, Devarajan P. Urine NGAL predicts severity of acute kidney injury after cardiac surgery: a prospective study. Clin J Am Soc Nephrol. 2008 May; 3(3):665-673. PMCID: PMC2386703
14. Zhou C, Gong J, Chen D, Wang W, Liu M, Liu B. Levosimendan for Prevention of Acute Kidney Injury After Cardiac Surgery: A Meta-analysis of Randomized Controlled Trials. Am J Kidney Dis. 2016 March;67(3):408-416. PMID: 26518388
15. Oh S W, Chin H J, Chae D W, Na K Y. Erythropoietin improves long-term outcomes in patients with acute kidney injury after coronary artery bypass grafting. J Korean Med Sci. 2012 May; 27(5):506-511. PMCID: PMC3342541
16. Tasanarong A, Duangchana S, Sumransurp S, Homvises B, Satdhabudha O. Prophylaxis with erythropoietin versus placebo reduces acute kidney injury and neutrophil gelatinase-associated lipocalin in patients undergoing cardiac surgery: a randomized, double-blind controlled trial. BMC Nephrol. 2013 Jul. 5; 14:136. PMCID: PMC3704968
17. Minneci P C, Deans K J, Zhi H, Yuen P S T, Star R A, Banks S M, Schechter A N, Natanson C, Gladwin MT, Solomon SB. Hemolysis-associated endothelial dysfunction mediated by accelerated NO inactivation by decompartmentalized oxyhemoglobin. J Clin Invest. 2005 December; 115(12):3409-3417. PMCID: PMC1283939
18. Billings F T, Ball S K, Roberts L J, Pretorius M. Postoperative acute kidney injury is associated with hemoglobinemia and an enhanced oxidative stress response. Free Radic Biol Med. 2011 Jun. 1; 50(11):1480-1487. PMCID: PMC3090463
19. Vermeulen Windsant I C, Snoeijs M G, Hanssen S J, Altintas S, Heijmans J H, Koeppel T A, Schurink G W H, Buurman W A, Jacobs M J. Hemolysis is associated with acute kidney injury during major aortic surgery. Kidney Int. 2010 May; 77(10):913-920. PMID: 20182411
20. Kim-Campbell N, Gretchen C, Callaway C, Felmet K, Kochanek P M, Maul T, Wearden P, Sharma M, Viegas M, Munoz R, Gladwin MT, Bayir H. Cell-Free Plasma Hemoglobin and Male Gender Are Risk Factors for Acute Kidney Injury in Low Risk Children Undergoing Cardiopulmonary Bypass. Crit Care Med. 2017 November;45(11):e1123-e1130. PMCID: PMC5657595
21. Eberhardt R T, McMahon L, Duffy S J, Steinberg M H, Perrine S P, Loscalzo J, Coffman J D, Vita J A. Sickle cell anemia is associated with reduced nitric oxide bioactivity in peripheral conduit and resistance vessels. Am J Hematol. 2003 October;74(2):104-111. PMID: 14508796
22. Reiter C D, Wang X, Tanus-Santos J E, Hogg N, Cannon R O, Schechter A N, Gladwin MT. Cell-free hemoglobin limits nitric oxide bioavailability in sickle-cell disease. Nat Med. 2002 Dec;8(12):1383-1389. PMID: 12426562
23. Kapralov A, Vlasova I I, Feng W, Maeda A, Walson K, Tyurin V A, Huang Z, Aneja R K, Carcillo J, Bayir H, Kagan V E. Peroxidase activity of hemoglobin-haptoglobin complexes: covalent aggregation and oxidative stress in plasma and macrophages. J Biol Chem. 2009 Oct. 30; 284(44):30395-30407. PMCID: PMC2781594
24. Yeo T W, Lampah D A, Tjitra E, Gitawati R, Kenangalem E, Piera K, Granger D L, Lopansri B K, Weinberg J B, Price R N, Duffull S B, Celermajer D S, Anstey N M. Relationship of cell-free hemoglobin to impaired endothelial nitric oxide bioavailability and perfusion in severe falciparum malaria. J Infect Dis. 2009 Nov. 15; 200(10):1522-1529. PMCID: PMC3740798
25. Christen S, Finckh B, Lykkesfeldt J, Gessler P, Frese-Schaper M, Nielsen P, Schmid E R, Schmitt B. Oxidative stress precedes peak systemic inflammatory response in pediatric patients undergoing cardiopulmonary bypass operation. Free Radic Biol Med. 2005 May 15;38(10):1323-1332. PMID: 15855050
26. Vermeulen Windsant I C, de Wit N C J, Sertorio J T C, van Bijnen A A, Ganushchak Y M, Heijmans J H, Tanus-Santos J E, Jacobs M J, Maessen J G, Buurman W A. Hemolysis during cardiac surgery is associated with increased intravascular nitric oxide consumption and perioperative kidney and intestinal tissue damage. Front Physiol. 2014; 5:340. PMCID: PMC4157603
27. Kim-Campbell N, Gretchen C, Ritov V B, Kochanek P M, Balasubramani G K, Kenny E, Sharma M, Viegas M, Callaway C, Kagan V E, Bayfr H. Bioactive Oxylipins in Infants and Children With Congenital Heart Disease Undergoing Pediatric Cardiopulmonary Bypass. Pediatr Crit Care Med. 2020 January;21(1):33-41. PMCID: PMC7388063
28. Lyu L, Long C, Hei F, Ji B, Liu J, Yu K, Chen L, Yao J, Hu Q, Hu J, Gao G. Plasma Free Hemoglobin Is a Predictor of Acute Renal Failure During Adult Venous-Arterial Extracorporeal Membrane Oxygenation Support. J Cardiothorac Vasc Anesth. 2016 Aug;30(4):891-895. PMID: 27238434
29. Gburek J, Verroust P J, Willnow T E, Fyfe J C, Nowacki W, Jacobsen C, Moestrup S K, Christensen E L. Megalin and cubilin are endocytic receptors involved in renal clearance of hemoglobin. J Am Soc Nephrol. 2002 February;13(2):423-430. PMID: 11805171
30. Sheerin N S, Sacks S H, Fogazzi G B. In vitro erythrophagocytosis by renal tubular cells and tubular toxicity by haemoglobin and iron. Nephrol Dial Transplant. 1999 Jun;14(6):1391-1397. PMID: 10382998
31. Chan W L, Tang N L, Yim C C, Lai F M, Tam M S. New features of renal lesion induced by stroma free hemoglobin. Toxicol Pathol. 2000 October;28(5):635-642. PMID: 11026597
32. Kino K, Mizumoto K, Watanabe J, Tsunoo H. Immunohistochemical studies on hemoglobin-haptoglobin and hemoglobin catabolism sites. J Histochem Cytochem. 1987 Mar;35(3):381-386. PMID: 3546486
33. Betrus C, Remenapp R, Charpie J, Kudelka T, Brophy P. Smoyer W E, Lin J-J. Enhanced hemolysis in pediatric patients requiring extracorporeal membrane oxygenation and continuous renal replacement therapy. Ann Thorac Cardiovasc Surg. 2007 Dec;13(6):378-383. PMID: 18292719
34. Skogby M, Mellgren K, Adrian K, Friberg L G, Chevalier J Y, Mellgren G. Induced cell trauma during in vitro perfusion: a comparison between two different perfusion systems. Artif Organs. 1998 December; 22(12):1045-1051. PMID: 9876097
35. Vieira F U, Costa E T, Vieira R W, Antunes N, Petrucci O, de Oliveira P P. The effect on hemolysis of the raceway profile of roller pumps used in cardiopulmonary bypass. ASAIO J. 2012 February;58(1):40-45. PMID: 21266907
36. Schaer D J, Buehler P W, Alayash Al, Belcher J D, Vercellotti G M. Hemolysis and free hemoglobin revisited: exploring hemoglobin and hemin scavengers as a novel class of therapeutic proteins. Blood. 2013 Feb. 21; 121(8):1276-1284. PMCID: PMC3578950
37. Omar H R, Mirsaeidi M, Socias S, Sprenker C, Caldeira C, Camporesi E M, Mangar D. Plasma Free Hemoglobin Is an Independent Predictor of Mortality among Patients on Extracorporeal Membrane Oxygenation Support. Frati G, editor. PLOS ONE. 2015 Apr. 22; 10(4):e0124034. PMCID: PMC4406730
38. Loscalzo J, Welch G. Nitric oxide and its role in the cardiovascular system. Prog Cardiovasc Dis. 1995 October;38(2):87-104. PMID: 7568906
39. Carlsen E, Comroe J H. The rate of uptake of carbon monoxide and of nitric oxide by normal human erythrocytes and experimentally produced spherocytes. J Gen Physiol. 1958 Sep. 20; 42(1):83-107. PMCID: PMC2194895
40. Liu X, Miller M J, Joshi M S, Sadowska-Krowicka H, Clark D A, Lancaster J R. Diffusion-limited reaction of free nitric oxide with erythrocytes. J Biol Chem. 1998 Jul. 24; 273(30):18709-18713. PMID: 9668042
41. Dalton H J, Cashen K, Reeder R W, Berg R A, Shanley T P, Newth C J L, Pollack M M, Wessel D, Carcillo J, Harrison R, Dean J M, Meert K L, Eunice Kennedy Shriver National Institute of Child Health and Human Development Collaborative Pediatric Critical Care Research Network (CPCCRN). Hemolysis During Pediatric Extracorporeal Membrane Oxygenation: Associations With Circuitry, Complications, and Mortality. Pediatr Crit Care Med. 2018 November; 19(11):1067-1076. PMCID: PMC6218309
42. Meyer C, Heiss C, Drexhage C, Kehmeier E S, Balzer J, Muhlfeld A, Merx M W, Lauer T, KUhl H, Floege J, Kelm M, Rassaf T. Hemodialysis-induced release of hemoglobin limits nitric oxide bioavailability and impairs vascular function. J Am Coll Cardiol. 2010 Feb. 2; 55(5):454-459. PMID: 20117459
43. Vallelian F, Pimenova T, Pereira C P, Abraham B, Mikolajczyk M G, Schoedon G, Zenobi R, Alayash Al, Buehler P W, Schaer D J. The reaction of hydrogen peroxide with hemoglobin induces extensive alpha-globin crosslinking and impairs the interaction of hemoglobin with endogenous scavenger pathways. Free Radic Biol Med. 2008 Oct. 15; 45(8):1150-1158. PMID: 18708138
44. Tanaka M, Suzuki Y, Shirato I, Takahara H, Shibata T, Sugaya T, Shimamoto K, Horikoshi S, Tomino Y. Tubular epithelial cells have the capacity to transdifferentiate into CD68-positive macrophage-like cells by oxidative stress. Inflamm Res. 2008 Dec;57(12):593-600. PMID: 19109740
45. Hogg N, Kalyanaraman B. Nitric oxide and lipid peroxidation. Biochim Biophys Acta. 1999 May 5;1411(2-3):378-384. PMID: 10320670
46. Hulko M, Kunz M, Yildirim M, Homeyer S, Amon O, Krause B. Cell-free plasma hemoglobin removal by dialyzers with various permeability profiles. Sci Rep. 2015 Dec;5(1). PMCID: PMC4639840
47. Gleason T G, Argenziano M, Bavaria J E, Kane L C, Coselli J S, Engelman R M, Tanaka K A, Awad A, Sekela M E, Zwischenberger J B. Hemoadsorption to Reduce Plasma-Free Hemoglobin During Cardiac Surgery: Results of REFRESH I Pilot Study. Semin Thorac Cardiovasc Surg. 2019; 31(4):783-793. PMID: 31085219
48. Poli E C, Alberio L, Bauer-Doerries A, Marcucci C, Roumy A, Kirsch M, De Stefano E, Liaudet L, Schneider A G. Cytokine clearance with CytoSorb® during cardiac surgery: a pilot randomized controlled trial. Crit Care. 2019 Apr. 3; 23(1):108. PMCID: PMC6448322
49. Raval J S, Wearden P D, Orr R A, Kiss J E. Plasma exchange in a 13-year-old male with acute intravascular hemolysis and acute kidney injury after placement of a ventricular assist device. J Clin Apher. 2012 November; 27(5):274-277. PMID: 22811253
50. Hayes C, Shafi H, Mason H, Klapper E. Successful reduction of plasma free-hemoglobin using therapeutic plasma exchange: A case report. Transfus Apher Sci. 2016 Apr;54(2):253-255. PMID: 26388049
51. Baek J H, D′Agnillo F, Vallelian F, Pereira C P, Williams M C, Jia Y, Schaer D J, Buehler PW. Hemoglobin-driven pathophysiology is an in vivo consequence of the red blood cell storage lesion that can be attenuated in guinea pigs by haptoglobin therapy. J Clin Invest. 2012 Apr;122(4):1444-1458. PMCID: PMC3314461
52. Kubota K, Egi M, Mizobuchi S. Haptoglobin Administration in Cardiovascular Surgery Patients: Its Association With the Risk of Postoperative Acute Kidney Injury. Anesth Analg. 2017 June;124(6):1771-1776. PMID: 28525506
53. Tanaka K, Kanamori Y, Sato T, Kondo C, Katayama Y, Yada I, Yuasa H, Kusagawa M. Administration of haptoglobin during cardiopulmonary bypass surgery. ASAIO Trans. 1991 Sep;37(3):M482-483. PMID: 1721515
54. Drabek T, Stezoski J, Garman R H, Wu X, Tisherman S A, Stezoski S W, Fisk J A, Jenkins L, Kochanek P M. Emergency preservation and delayed resuscitation allows normal recovery after exsanguination cardiac arrest in rats: a feasibility trial. Crit Care Med. 2007 Feb;35(2):532-537. PMID: 17205010 55. Miles L F, Coulson T G, Galhardo C, Falter F. Pump Priming Practices and Anticoagulation in Cardiac Surgery: Results From the Global Cardiopulmonary Bypass Survey. Anesth Analg. 2017 December; 125(6):1871-1877. PMID:
56. Kim S, Ye S, Adamo A, Orizondo R A, Jo J, Cho S K, Wagner W R. A biostable, anti-fouling zwitterionic polyurethane-urea based on PDMS for use in blood-contacting medical devices. J Mater Chem B. 2020 September;8(36):8305-8314. PMID: 32785384
57. Malkin A D, Ye S-H, Lee E J, Yang X, Zhu Y, Gamble L J, Federspiel W J, Wagner W R. Development of zwitterionic sulfobetaine block copolymer conjugation strategies for reduced platelet deposition in respiratory assist devices. J Biomed Mater Res B Appl Biomater. 2018 March; PMCID: PMC6085169
58. Protein A-Agarose, Protein G-Agarose: Affinity chromatography matrix for purification of antibodies and for the immunoprecipitation of proteins. Package Insert. Roche; 2016.

What is claimed is:

1. An extracorporeal hemoadsorption device comprising: a purification column, wherein said purification column comprises a capture structure, wherein a ligand is conjugated on a surface of said capture structure, wherein said ligand is specific for a component to be removed from blood of the subject.

2. The device of claim 1, wherein said device is connected to a device for extracorporeal therapy, wherein said extracorporeal therapy removes fluid from a subject and returns fluid to the same subject.

3. The device of claim 2, wherein the extracorporeal therapy is an extracorporeal life support (ECLS) system.

4. The device of claim 3, wherein the ECLS system comprises a pump.

5. The device of claim 2, wherein the device for extracorporeal therapy is a cardiopulmonary bypass system.

6. The device of claim 2, wherein the device for extracorporeal therapy is an extracorporeal membrane oxygenation (ECMO) system.

7. The device of claim 2, wherein the device for extracorporeal therapy further comprises an oxygenator.

8. The device of claim 2, wherein the device for extracorporeal therapy further comprises a venous reservoir.

9. The device of claim 2, wherein the device for extracorporeal therapy is a ventricular assist device.

10. The device of claim 2, wherein the device for extracorporeal therapy involves plasma exchange.

11. The device of claim 2, wherein the device for extracorporeal therapy is a renal replacement therapy device.

12. The device of any one of claims 1-11, wherein the capture structure is a porous bead.

13. The device of claim 12, wherein the porous bead is between 50 and 500 μm.

14. The device of claim 12 or 13, wherein the bead further comprises a polymer attached to the bead.

15. The device of claim 14, wherein the polymer is polyethylene glycol (PEG).

16. The device of any one of claims 1-15, wherein the specific component to be removed comprises a protein.

17. The device of claim 16, wherein the protein is cell-free plasma hemoglobin.

18. The device of claim 17, wherein the ligand is zinc, haptoglobin, or hemoglobin-binding protein (HBP).

19. The device of claim 16, wherein said protein is an antibody, cytokine, or toxin.

20. The device of any one of claims 1-19, wherein the component to be removed from the blood is a virus or bacteria.

21. The device of any one of claims 1-20, wherein the device is configured so that blood enters the hemoadsorption device at a proximal end and blood exits the hemoadsorption device at a distal end.

22. The device of claim 21, wherein said proximal end of the hemoadsorption device is in fluid communication with a pump.

23. The device of claim 12, wherein the porous bead is incorporated in a cartridge.

24. The device of any one of claims 1-23, wherein said device comprises an integrated flow sensor.

25. The device of any one of claims 1-24, wherein said device comprises an integrated pressure differential sensor.

26. The device of claim 25, wherein the device further comprises an integrated mechanism to adjust resistance to allow modification of blood flow through the device.

27. The device of any one of claims 1-26, wherein the device comprises one or more sensors used to measure cell-free hemoglobin.

28. The device of claim 27, wherein the device measures overall systemic levels of cell-free plasma hemoglobin.

29. The device of claim 27, wherein the device measures overall saturation of cell-free plasma hemoglobin within the device, thereby requiring a cartridge change.

30. A method for removing a target component from a subject, the method comprising:
a. removing blood from a subject; and
b. exposing said blood from step a) to a purification column, wherein said purification column comprises at least one capture structure, wherein a ligand is conjugated on a surface of said capture structure, wherein said ligand is specific for a component to be removed from the blood of the subject, thereby removing said component from the subject's blood; and
c. returning the blood to the subject.

31. The method of claim 30, wherein said blood is removed and returned to the subject via a pump.

32. The method of claim 31, wherein said pump is part of an extracorporeal therapy system.

33. The method of claim 32, further comprising oxygenating the blood.

34. The method of claim 32 or 33, wherein the method further comprises passing the blood through a venous reservoir.

35. The method of any one of claims 32-34, wherein the extracorporeal therapy system is a cardiopulmonary bypass system.

36. The method of any one of claims 32-35, wherein the extracorporeal therapy system is a ventricular assist device.

37. The method of any one of claims 32-36, wherein the extracorporeal therapy system is a renal replacement therapy device.

38. The method of any one of claims 32-37, wherein the capture structure is a porous bead.

39. The method of claim 38, wherein the bead is between 50 and 500 μm.

40. The method of claim 38 or 39, wherein the bead further comprises a polymer attached to the bead.

41. The method of claim 40, wherein the polymer is polyethylene glycol (PEG).

42. The method of any one of claims 30-41, wherein the target to be removed is a protein.

43. The method of claim 42, wherein the protein to be removed is cell-free plasma hemoglobin.

44. The method of claim 43, wherein the ligand is zinc, haptoglobin, or hemoglobin-binding protein (HBP).

45. The method of claim 43, wherein the protein is an antibody, cytokine, or toxin.

46. The method of any one of claims 30-45, wherein the component to be removed from the blood is a virus or bacteria.

47. A system comprising:
a) a purification chamber comprising:
a purification column, wherein said purification column comprises a capture structure, wherein a ligand is conjugated on a surface of said capture structure, wherein said ligand is specific for a component to be removed from a fluid of a subject; and
b) a pressure source configured to circulate the fluid from a subject through the purification chamber.

48. The system of claim 47, wherein the purification chamber further comprises an inlet and an outlet, wherein the fluid flows from the outlet to the inlet.

49. The system of claim 47 or 48, further comprising a sensor configured to measure a property of the fluid.

50. The system of claim 49, wherein the property of the fluid is a hemoglobin concentration of the fluid.

51. The system of claim 49 or claim 50, wherein the sensor is configured to measure absorbance at approximately 540 nm.

52. The system of any one of claims 49-51, wherein the sensor is an optical sensor configured to measure a property of the fluid entering the inlet of the purification chamber.

53. The system of any one of claims 49-52, wherein the sensor is an optical sensor configured to measure a property of the fluid entering the outlet of the purification chamber.

54. The system of any one of claims 47-53, further comprising a regeneration chamber in fluid communication with the purification chamber.

55. The system of any one of claims 47-53, wherein the regeneration chamber comprises guanidine.

56. The system of claim 52 further wherein the regeneration chamber comprises a removable cartridge.

57. The system of any one of claims 47-56, further comprising a bypass conduit in fluid communication with the pressure source and the subject.

58. The system of any one of claims 47-57, further comprising an oxygenator in fluid communication with the pressure source.

59. The system of any one of claims 47-58, further comprising a venous reservoir in fluid communication with the pressure source.
